(19) United States
(12) Patent Application Publication
(10) Pub. No.: US 2017/0181955 A1
(43) Pub. Date: Jun. 29, 2017

(54) COMPOSITION FOR SHAVING

(71) Applicant: NITSAN PRIMOR, TEL AVIV (IL)

(72) Inventor: NITSAN PRIMOR, TEL AVIV (IL)

(21) Appl. No.: 15/300,285

(22) PCT Filed: Mar. 15, 2015
(86) PCT No.: PCT/IL2015/050270

§ 371 Date: Sep. 29, 2016

Related U.S. Application Data
(60) Provisional application No. 61/977,110, filed on Apr. 9, 2014.

(51) Int. Cl. A61K 8/86; A61K 8/37; A61K 8/34; A61K 8/31; A61Q 9/02; A61K 8/92; A61K 8/49; A61K 8/81; A61K 8/33; A61Q 19/00; A61K 8/19

(52) U.S. Cl. CPC A61K 8/86; A61Q 19/007; A61K 8/37; A61K 8/342; A61K 8/31; A61K 8/34; A61K 8/19; A61K 8/922; A61K 8/498; A61K 8/4913; A61K 8/8182; A61K 8/33; A61Q 9/02; A61K 2800/75; A61K 2800/48

(57) ABSTRACT

The invention provides a non-greasy skin conditioning composition and related wipes that may be used as a pre-shave and/or after-shave composition with enhanced lubricity and balanced humidity along with an optional odorant that obviates the use of any aftershave.

### RELATED APPLICATIONS

This application claims the benefit of U.S. Provisional Patent Application No. 61/977,110 filed on Apr. 9, 2014.

### FIELD OF THE INVENTION

The present invention generally relates to cosmetic compositions, more particularly cosmetic compositions for conditioning skin when shaving and wipes especially designed to be used in connection with razors and electric shavers.

### BACKGROUND OF THE INVENTION

When consumers use an electric razor, they can sense the friction between the skin and the razor head. Friction can lead to uncomfortable skin irritation. But more importantly, friction between the razor head and skin will distort the skin in front of the razor causing it to “bunch up” or create “a wave.” This effectively changes the angle of the razor head to the beard hair being cut and thereby greatly reduces the efficiency of the shaving process.

Compositions applied to the skin prior to shaving generally seek to prepare the skin to receive a razor, providing lubrication for reducing friction to prevent irritation or cutting of the skin. With respect to preparations designed for electric shaving, typically, these compositions have the disadvantage of leaving the skin feeling greasy or oily, which is uncomfortable and unpleasant to the user.

Many have attempted to increase the lubricity of pre-shave compositions by including high oil contents therein. These compositions have the typical drawbacks of high-oil vehicles, namely leaving a residue of oil on the skin, causing the skin to feel oily or greasy, which is unpleasant or uncomfortable to the user and may require additional treatment for the skin, such as washing or wiping with an oil-reducing toner to remove any oily residue.

To reduce these undesirable effects, some pre-shave compositions incorporate plate-form powders, such as talc. Plate-form powders have the characteristic of adhering to the skin or electric razor. However, powders dehydrate skin moisture, causing dryness. Other pre-shave compositions incorporate globular powders, such as nylon, polystyrene, polyethylene, and polyester, which are spherical in shape. One deficiency with solely focusing on lubricity is that there is not a direct correlation between improved lubricity and closeness of shaving.

Numerous types of after shave products are known. Many of these products have a very high alcohol content (e.g., >50%), although some have low alcohol or no alcohol. Each type of formulation has its drawbacks. The high alcohol formulations tend to cause dryness, stinging and a burning sensation, while the low to no alcohol formulations tend to dry too slowly and feel wet. None of these products reduces redness and irritation.

Accordingly, shave-related compositions for use before or after shaving, e.g., with an electric shaver, are required to overcome the above-mentioned problems and drawbacks.

Topical wipes are well known for a variety of applications, including topical application to human skin or other applied surfaces to provide such surfaces with cleansing, moisturizing, depositing of active ingredients or other materials, disinfecting, and/or any other effect that can be accomplished by application of a topical wipe to the desired surface.

Lanthanum is known to have pharmacological effects on several receptors and ion channels in the human body and has specificity for the GABA receptor. Lanthanum acts at the same modulatory site on the GABA receptor as zinc, which is a known negative allosteric modulator. The lanthanum cation La³⁺ is a positive allosteric modulator at native and recombinant GABA receptors, increasing open channel time and decreasing desensitization in a subunit configuration dependent manner (Boldyreva, A. A., “Lanthanum Potentiates GABA-Activated Currents in Rat Pyramidal Neurons of CA1 Hippocampal Field”. Bulletin of Experimental Biology and Medicine 140 (4): 403-5, 2005; Zubrzycka M, Janecka A., “Substance P: transmitter of nociception (Mini-review)”. Endocrine Regulations 34 (4): 195-201, 2000).

### SUMMARY OF THE INVENTION

The present invention relates to an all-in-one shaving-related composition for use in shaving, i.e., before, during and/or after shaving, particularly with an electric shaver. The invention provides a non-greasy skin conditioning composition that may be used as a pre-shave and/or after-shave composition with enhanced lubricity and balanced humidity, along with an optional odorant that obviates the use of any aftershave. The composition of the invention also has disinfectant properties to prevent skin infection and anti-allergic properties to prevent or reduce irritation. The composition thus provides an effective, efficient and pleasing shaving experience to the user, leaving the skin soft, moisturized and with a pleasant scent.

In an aspect of the present invention, there is provided a skin conditioning composition formulated to lubricate skin and impart emollient or moisturizing properties before, during and after shaving.

In another aspect of the present invention, there is provided a skin conditioning composition formulated to reduce skin friction between a razor and a skin surface.

The skin conditioning composition of the invention is adapted for use in shaving, preferably before or after shaving with an electric shaver. The composition comprises a lubricating agent, or mixture of such agents, and one or more aqueous-soluble trivalent cation from the lanthanide series, preferably aqueous-soluble trivalent lanthanide cation in the form of salt. Surprisingly, lanthanide salts have anti-irritant properties that are especially beneficial in the shaving compositions of the invention.

More specifically, the composition comprises 1%-80% by weight alcohol, 0.1-10% by weight lubricating agent and 0.1-5% by weight anti-irritant agent comprising one or more aqueous-soluble trivalent cation of the lanthanide series.

The trivalent cation is preferably selected from La³⁺, Ce³⁺, Pr³⁺, more preferably La³⁺ in the form of salt or hydrated salt, and most particularly lanthanum nitrate. The composition also comprises additional anti-irritant agents selected from bisabolol, tocopherol and saw palmetto extract.

The composition may further comprise 0.1-5% by weight of a low molecular weight polyethylene glycol (PEG) as a lubricant, 0.1-5% by weight of a thickener agent and at least one additional ingredient selected from the group consisting of skin conditioner, humectant, antiseptic agent, anti-allergic agent, color, fragrance, antioxidant, natural extract, vitamin, UV light absorber, solvent and combinations of these.

The composition can be in the form of a solution, emulsion, spray, gel or in solid form, such as stick or roll-on. Alternatively, the composition can be impregnated within a physical applicator selected from a wipe, pad, towelette, cloth, tissue, swab and bandage, hereinafter collectively termed “wipe” or “wet wipe”.

A wipe according to the invention is intended for application of the composition onto a user's skin before, during or after shaving. It is made of a flexible substrate made of an absorbent material and has a skin conditioning composition impregnated therein or applied thereon, the skin conditioning composition comprising a lubricating agent, or mixture of such agents, and one or more aqueous-soluble trivalent lanthanide cations that have beneficial anti-irritant properties. Preferably, the trivalent cation of the lanthanide series is selected from La³⁺, Ce³⁺ and Pr³ and salts of any of these cations. More preferably, the trivalent cation of the lanthanide series is a trivalent salt of La³⁺, such as, for example, lanthanum nitrate La(NO₃)₃) or lanthanum nitrate hexahydrate.

In a preferred embodiment, the skin conditioning composition within the wipe further comprises at least one additional agent selected from the group consisting of a skin conditioner, humectant, antiseptic agent, anti-allergic agent, color, fragrance, antioxidant, natural extract, vitamin, JV light absorber, solvent and combinations of these.

In another preferred embodiment, the skin conditioning composition within the wipe comprises lanthanum nitrate or lanthanum nitrate hexahydrate, alcohol, tocopherol, bisabolol and saw palmetto extract.

In a preferred embodiment, the wipe is intended for use before shaving, particularly before shaving with an electric shaver, thereby leaving a layer on a user's skin surface for facilitating shaving, with the proviso that the layer is not in the form of foam and does not form foam upon application on the skin.

The composition and corresponding wipe of the invention condition the skin before, during and after shaving by providing at least one of the following properties to the skin: enhanced lubricity, balanced humidity, disinfectant properties and anti-irritant properties.

In another aspect, the invention provides a method of conditioning a skin area before shaving, the method comprising applying the wipe or the composition of the invention to a hairy skin area, for example, the skin of the face, neck, scalp, arm pits, legs, in an amount effective to prepare the area to be shaved. The composition of the present invention is particularly effective when the shaving is by electric razor.

In another aspect, the invention provides a method of conditioning a skin area after shaving, the method comprising applying the wipe or the composition of the invention to a shaved skin area in an amount effective to condition the shaved skin in that area.

In another preferred embodiment, the method contemplates applying the wipe or the composition of the invention just before the shaving or sufficient time before the shaving to afford a smooth shave, e.g., 2-20 seconds before shaving.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### DESCRIPTION OF SPECIFIC EMBODIMENTS OF THE INVENTION

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Thus, according to an aspect of the present invention there is provided an all-in-one pre- and/or after-shave composition that makes shaving smooth, leaving the skin soft, moisturized, with a pleasant odor and with minimal irritation. Preferably, the composition of the invention is suitable for use before, during and/or after shaving with an electric shaver.

The skin conditioning composition may be used as a pre-shave and/or after-shave composition with enhanced lubricity and balanced humidity along with an optional odorant that obviates the use of any aftershave. The composition of the invention also has disinfectant properties to prevent skin infection and anti-allergic properties to prevent irritation.

The composition of the invention conditions the skin in shaving, especially before shaving with an electric shaver, by providing at least one of the following properties to the skin: enhanced lubricity, balanced humidity, disinfectant properties and anti-irritant properties.

The composition comprises a lubricating agent, or a mixture of such agents, and one or more aqueous-soluble trivalent cations of the lanthanide series in the form of salts, preferably a lanthanide salt. Surprisingly, these lanthanide salts have anti-irritant properties that are especially beneficial in the composition of the invention when applied before or after shaving.

Anti-irritant agents are intended to reduce the irritation potential of other agents that may cause skin irritation, sufficiently to be tolerated when such irritants are applied to the body. Different mechanisms are postulated for the activity shown by various anti-irritants: some operate by complexing the irritant itself, others react with the skin, blocking reactive sites so that the irritant cannot react with it, and another group seems to protect by preventing complete physical contact between irritant and skin.

A lubricating agent for purposes of this invention is a substance that reduces friction between the surface of a razor blade in contact with a skin surface.

The skin conditioning composition of the invention comprises a trivalent cation of the lanthanide series selected from La³⁺, Ce³⁺ and Pr³, and salts of the foregoing cations. Preferably, the trivalent cation is La³⁺ in the form of a salt, such as, for example, lanthanum nitrate or lanthanum nitrate hexahydrate. The composition may additionally comprise anti-irritation agents selected from a long chain fatty alcohol, tocopherol, bisabolol and saw palmetto extract.

The skin conditioning composition of the invention is adapted for use before or after shaving, particularly before or after shaving with an electric shaver. The composition comprises l%-80% by weight volatile agent, preferably a liquid alcohol, 0.1-10% by weight lubricating agent, 0.1-5% by weight lanthanum salt and at least one anti-irritant agent selected from, bisabolol, tocopherol, saw palmetto extract and combinations of any of these anti-irritant agents.

The composition may further comprise 0.1-5% by weight of a low molecular weight polyethylene glycol (PEG), 0.1-5% by weight of a thickener agent and/or at least one additional ingredient selected from the group consisting of skin conditioner, humectant, antiseptic agent, anti-allergic agent, color, fragrance, antioxidant, natural extract, vitamin, UV light absorber, solvent and combinations of these.

The composition can be in the form of a solution, emulsion, spray, gel or in solid or semi-solid form, such as stick or roll-on. Alternatively, the composition can be impregnated within or applied onto a physical applicator, preferably a flexible substrate selected from wipe, pad, towelette, cloth, tissue, swab and bandage, hereinafter collectively termed “wipe” or “wet wipe”.

The flexible substrate is preferably manufactured from an absorbent or a porous material. The use of an absorbent or porous material advantageously assists in maintaining the composition in or on the substrate.

The substrate may include, but is not limited to, non-woven substrates and woven substrates. The term ‘non-woven’ means that the substrate, or at least a layer of the substrate, is formed of fibers which are not woven into a fabric and are, therefore, formed into a sheet, mat, or pad layer. The fibers can either be random (i.e., randomly aligned) or they can be carded (i.e., combed to be oriented in primarily one direction). Furthermore, the non-woven substrate can include a combination of layers of random and carded fibers.

When the substrate is a woven substrate, it is envisaged that it may include woven cotton and polyester substrates.

It is particularly preferred that the substrate is in the shape of a substantially flat sheet, such as a wipe, towelette, pad, cloth, tissue, swab or the like. It is envisaged that the shape of the substrate may be, for example, square, circular, oval, or rectangular.

Production of the wipes can be performed in a special dedicated machine in which the roll of the fabric is conveyed into a pool containing the composition. The wet fabric is then cut into pieces. The machine folds the wet wipe and packs it in a hermetic closed aluminum sachet.

Alternatively, the composition of the invention may be applied in solid form within a stick or in solid, semi-solid or liquid form in a roll-on device.

According to embodiments of the invention, the wipe comprises the skin conditioning composition of the invention for applying on a user's skin to be shaved, thereby leaving a. layer on the skin surface for facilitating shaving, with the proviso that the layer is not in the form of foam and does not form foam upon application on the skin, thus being suitable for use before any shave, especially before shaving with an electric razor. In accordance with embodiments of the present invention, a skin conditioning composition is provided which is non-greasy to the skin and touch and which forms a layer on the skin surface for facilitating shaving. The layer is preferably non-foaming. The skin conditioning composition of the invention, which may be in the form of a solution, an emulsion, a spray, gel or the like, may comprise one or more skin-conditioning agents and/or skin feel enhancers and/or humectants, one or more lubricants, one or more disinfectants, one or more anti-allergic agents, optionally one or more preservatives, and water. The composition of the invention also optionally includes one or more colorants and/or fragrances.

As indicated, the composition of the invention includes a unique combination of ingredients that aids in forming a skin-conditioning composition designed to be used pre shave and after shave, which combination comprises ingredients which are beneficial in general as skin-conditioning agents for the pre-shave step and for the after-shave step.

As formulated for use with electric or regular razors, the composition of the invention incorporates volatile agents that impart a drying ability to the pre-shave aspect. Preferably these volatile agents are liquid alcohols, such as ethyl alcohol and isopropyl alcohol. The amount of volatile agent in the composition is at least 1% by weight, preferably at least 20% by weight, more preferably at least 50% by weight, most preferably 70% or 80% per weight. These ranges ensure desirable drying characteristics of the composition before the application of a razor. The high volatility of the volatile agent stiffens the beard hairs, making them stand-up more readily and thus permits easier cutting.

The composition also contains various lubricants and moisturizers or emollients. Suitable lubricants and moisturizers or emollients include hydrocarbons, silicones, fatty alcohols, fatty acids, synthetic or natural esters, combinations thereof and the like. Suitable lubricants and moisturizers or emollients can be included in an amount between about 0.1% to about 20%, preferably in an amount ranging from about 0,1% to about 15%, more preferably from about 0.1% to about 10%. Non limiting examples of lubricant and moisturizers or emollient agents that can be used in the composition of the invention are isopropyl palmitate, octyldodecanol, octyl alcohol, decyl alcohol, dodecyl alcohol, tetradecyl alcohol, cetyl alcohol, eicosyl alcohol, bis-PEG-18 methyl ether dimethylsilane, C12-15 alkylbenzoates, polyethylene glycols, siloxan derivatives, e.g., cyclic or linear dimethylpolysiloxanes, silicon polymers and copolymers, and many others.

Other ingredients suitable for use in the composition of the invention include, but are not limited to, skin conditioners, humectants, color, fragrance, antioxidants, natural extracts, vitamins, UV light absorbers, solvents, surfactants and combinations thereof.

Natural extracts suitable for use in the composition of the invention include extracts of saw palmetto, derivatives thereof and of components being part of said extract. The composition according to the present invention advantageously comprises saw palmetto extract, from 0.01-20.0%, preferably between 0.01-10% and more preferably between 0.1-5% by weight.

Water is employed in the composition of the invention as a solvent and carrier and will be present in an amount within the range of from about 10% to about 70% by weight.

Skin conditioning agents with efficient anti-irritant effects which are present in the composition of the invention include trivalent cations of the lanthanide series, selected from La³⁺, Ce³⁺ and Pr³, and salts of the foregoing cations. Such conditioning agent may be present in an amount within the range of from about 0.1-20%, preferably from about 0.1 to about 15%, more preferably from 0.1% to about 5% by weight.

The anti-irritant effects of the lanthanide cations can be optimized by suitable selection of the accompanying anionic species. Especially preferred cation-anion pairs include chloride and nitrate salts of the lanthanides. The appropriate cation concentration can be achieved using a single metal cation species, or multiple different cation species may be combined to yield the total desired cation concentration. A preferred anti-irritant agent is lanthanum nitrate. Lanthanum nitrate may be present in free salt form or as hydrate.

Skin conditioning agents with efficient anti-irritant action which may be present in the composition of the invention also include bisabolol from synthetic or natural origin. However, the bisabolol used in the context of the present invention can principally be of natural origin, and is preferably alpha-bisabolol. If natural bisabolol is used, this can also be employed as a constituent of an essential oil or of a plant extract or of a fraction thereof.

Further skin conditioning agents with anti-irritant action which may be present in the composition of the invention also include limonene, linalool, tocopherol and others. Such conditioning agents may be present in an amount within the range of from about 0.1-10%, preferably from about 0.1 to about 5% by weight.

The composition of the present invention may further include a detergent. Detergents most commonly used are known as amphoteric surfactants. An exemplary surfactant used in the composition of the invention is Cocoyl proline preferably from vegetable origin. Cocoyl proline is a lipoaminoacid obtained by acylation of fatty acids from coconut with proline and is used as surfactant in order to solubilize essential oils and fragrances.

Humectants may be added to prevent premature drying of the solution and contribute to skin pores contraction.

Other ingredients may include preservatives, to ensure the solution does not support microbial growth.

The skin preparations may include, in addition to the ingredients set out above, certified water-soluble colorants as deemed necessary, fragrances in amounts within the range of from about 0 to about 5% and preferably from about 0.1 to about 3% by weight, depending upon the ultimate use of the skin preparation, solubilizing agents, and the like.

The composition of the invention may also optionally include from 0% to about 10% by weight of one or more sun screen agents.

The composition may also include 0.1-5% of thickener and/or gelling agent for aqueous or oil-in-water emulsions. Thickeners are used to control the viscosity of the finished product and keep it the right consistency. Examples of suitable thickeners include sodium acrylate/sodium acryloyldimethyl taurate copolymer, acrylamide/sodium acryloyldirnethyltaurate copolymer, ammonium polyacryloyldimethyl taurate, ammonium acryloydimethyl taurate/VP copolymer, and combinations thereof. Preferred are the materials available under the tradename Aristoflex AVC™ from the company Clariant, consisting of ammonium acryloydimethyl taurate/VP copolymer.

The composition may also contain cosmetically acceptable additives or adjuvants as well as cosmetic or dermatologic active agents. Additives and adjuvants may be present in the compositions in amounts generally ranging from about 0.01% to about 10% by weight. These ingredients may be soluble or dispersible in whatever phase or phases is/are present in the composition.

The Preparation Method:

The ingredients used in the composition according to the invention that may be applied by a wipe may be prepared in large batch tanks. Depending on the formula requirements, the tank is charged with the first ingredient, which is usually water. The tank may be heated during manufacturing to facilitate blending of powders that must be dissolved or other solids that must be melted. The other ingredients are added sequentially and mixed until homogenous. An emulsion or other homogeneous solution is formed and is moved to the wipes automatic machine which is based on a “wet laid process”. In this wet process, the rollers of the fabric are soaked in the bath. These rolls are then further processed and slit into narrow widths and then perforated or cut into individual sheets. The finished sheets are then automatically folded, stacked, and transferred into sachet so that they can easily be removed.

### EXAMPLES

The following table depicts some non-limiting examples of pre- and after-shave compositions according to embodiments of the present invention:

TABLE 1

| Ingredient in % w/w | Example 1 | Example 2 | Example 3 | Example 4 | Example 5 | Example 6 |
|---|---|---|---|---|---|---|
| ALCOHOL | 50 | 60 | 65 | 50 | 25 | 35 |
| ISOPROPYL | 17 | 10 | 13 | 20 | 25 | 35 |
| PALMITATE | | | | | | |
| OCTYLDODECANOL | 1.6 | 2.0 | 1.0 | 2.5 | | |
| BIS-PEG-18 METHYL | 1.0 | 1.5 | 1.0 | 2.0 | 1.5 | 1.5 |
| ETHER DIMETHYL | | | | | | |
| SILANE | | | | | | |
| FRAGRANCE | 0.5 | 0.5 | 0.5 | 0.5 | 0.5 | 0.75 |
| LIMONENE | 0.5 | 0.5 | 0.5 | 0.5 | 0.5 | 0.5 |
| LINALOOL | 0.5 | 0.5 | 0.5 | 0.5 | 0.5 | 0.5 |
| LANTHANUM | 2.5 | 1.5 | 2.0 | 3.0 | 3.5 | 4.0 |
| NITRATE | | | | | | |
| SAW PALMETTO | 3.0 | 3.0 | 3.0 | 3.0 | 5.0 | 4.0 |
| TOCOPHEROL | 0.004 | 0.004 | 0.004 | 0.004 | 1.0 | 0.5 |
| BISABOLOL | 1.0 | 2.0 | 3.0 | 4.0 | 5.0 | 0.5 |
| COCOYL PROLYNE | 3.0 | 3.5 | 4.0 | 4.5 | 5.5 | 5.0 |
| ACRYLOYL | 1.0 | 1.5 | 1.0 | 2.0 | 1.0 | 1.0 |
| DIMETHYLTAURAT | | | | | | |
| E/VP COPOLYMER | | | | | | |
| C12-15 | 2.0 | 2.0 | 2.0 | 2.0 | 2.0 | 2.0 |
| ALKYLBENZOATE | | | | | | |
| HEXYL CINNAMAL | 0.0001 | 0.0001 | 0.0001 | 0.0001 | 0.0001 | 0.0001 |
| PRESERVATIVE | 0.1 | 0.1 | 0.1 | 0.1 | 0.1 | 0.1 |
| WATER | Ad 100 | Ad 100 | Ad 100 | Ad 100 | Ad 100 | Ad 100 |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. (canceled)

2. (canceled)

3. (canceled)

4. (canceled)

5. The physical applicator of claim 12, wherein said anti-irritant agent further comprises an agent selected from bisabolol, tocopherol, saw palmetto extract and combinations of any of these anti-irritant agents.

6. The physical applicator of claim 12, wherein said composition further comprises 0.1-5% by weight of a low molecular weight polyethylene glycol (PEG).

7. The physical applicator of claim 12, wherein said composition further comprises a thickener agent in an amount of 0.1-5% by weight.

8. (canceled)

9. (canceled)

10. The physical applicator of claim 12 wherein said composition is in the form of solution, emulsion, spray, gel or in solid or semi-solid form such as stick or roll-on.

11. The the physical applicator of claim 12, wherein said composition is impregnated in or applied to a physical applicator selected from wipe, pad, towelette, cloth, tissue, swab and bandage.

12. A physical applicator for a composition for use in shaving, the applicator comprising:
a flexible substrate made of an absorbent material; and
a skin conditioning composition impregnated in or applied on said flexible substrate, the skin conditioning composition comprising 1%-80% by weight volatile agent, 0.1-10% by weight lubricating agent and 0.1-10% by weight anti-irritant agent comprising one or more aqueous-soluble trivalent cation of the lanthanide series, the wipe intended for application on a user's skin before or after shaving.

13. (canceled)

14. (canceled)

15. The physical applicator of claim 12, wherein said trivalent cation of the lanthanide series is selected from La³⁺, Ce³⁺ and Pr³⁺ and salts of any of these cations.

16. The physical applicator of claim 12, wherein said trivalent cation of the lanthanide series is in the form of a trivalent salt of La³⁺.

17. The physical applicator of claim 12, wherein said trivalent cation of the lanthanide series is in the form of lanthanum nitrate (La(NO₃)₃) or lanthanum nitrate hexahydrate.

18. The method according to claim 23, further comprising applying said composition on a user's skin to be shaved thereby leaving a layer on the skin surface for facilitating shaving, with the proviso that the layer is not in the form of foam and does not form foam upon application on the skin.

19. The physical applicator of claim 12, wherein said skin conditioning composition further comprises at least one additional agent selected from the group consisting of skin conditioner, humectant, antiseptic agent, anti-allergic agent, color, fragrance, antioxidant, natural extract, vitamin, UV light absorber, solvent and combinations of these.

20. The physical applicator of claim 12, wherein said skin conditioning composition comprises 0.01-10.0% w/w lanthanum nitrate or lanthanum nitrate hexahydrate, 1-70% w/w alcohol, 0.01-5.0% w/w tocopherol, 0.01-10.0% w/w bisabolol and 0.01-10.0% w/w saw palmetto extract.

21. (canceled)

22. (canceled)

23. A method of conditioning a skin area before or after shaving comprising applying the physical applicator of claim 12 to a hairy skin area so as to apply the composition in an amount effective to prepare the area to be shaved.

24. The method according to claim 23, wherein the shaving is by electric razor.

25. A method of conditioning a skin area after shaving comprising applying the physical applicator of claim 12 to a hairy skin area so as to apply the composition in an amount effective to condition the shaved skin in that area.

26. The method of claim 23, wherein the composition is applied to the skin at least 2 seconds before shaving to reduce friction between an electric razor and a skin surface.

27. (canceled)

28. (canceled)

29. (canceled)

30. The method of claim 25, wherein the composition is applied to the skin at least 2 seconds before shaving to reduce friction between an electric razor and a skin surface.
